(19) United States
(12) Patent Application Publication
(10) Pub. No.: US 2021/0403839 A1
(43) Pub. Date: Dec. 30, 2021

(54) Skin Cleansing System and Method for Visual Indication of an Efficacious Cleansing Duration

(71) Applicant: Hanna Zane Masri, Bellaire, TX (US)

(72) Inventor: Hanna Zane Masri, Bellaire, TX (US)

(21) Appl. No.: 16/913,901

(22) Filed: Jun. 26, 2020

(51) Int. Cl. C11D 17/04; A47K 5/13; C11D 9/44; C11D 9/38; C11D 9/24; C11D 9/36; C11D 9/00

(52) U.S. Cl. CPC C11D 17/046; A47K 5/13; C11D 9/442; C11D 9/005; C11D 9/38; C11D 9/24; C11D 9/36; C11D 9/444

(57) ABSTRACT

A skin cleansing system and method for visual indication of an efficacious cleansing duration provides a cleaning composition that works to kill germs; and then visually indicate, through color, when a sufficient cleaning duration, of approximately 20 seconds, of cleansing has occurred. The cleaning composition comprises a liquid soap and a colored moisturizer that remain separated in a dispenser, but combine simultaneously when applied to the skin for washing with water. The soap requires a cleaning duration of about 20 seconds to kill the germs. The soap also requires the same cleaning duration of 20 seconds to dissolve the nonpolar oils in the colored moisturizer. Thus, when the colored moisturizer washes away, this is an indication that the germs have also washed away. The dissolving action is a result of the nonpolar tail of the soap engaging and destroying, both the germs and the nonpolar oils in the colored moisturizer.

### FIELD OF THE INVENTION

The present invention relates generally to a skin cleansing system and method for visual indication of an efficacious cleansing duration. More so, the present invention relates to a skin cleansing system comprising a cleaning composition consisting of a soap and a colored moisturizer, which works to kill and wash away germs; and then visually indicate, through color, when a sufficient duration of approximately 20 seconds of cleansing has occurred by simultaneously washing away a coloring agent in the moisturizer.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

It is recognized in the art that, with the Coronavirus rapidly spreading across the world, many people are asking what they can do to slow the spread of the highly infectious disease. For example, the World Health Organization has strongly advised the general populace to regularly and thoroughly wash their hands. It is also axiomatic that the hands should be washed for 20 seconds to properly clean them.

Generally, proper hand washing is effective for reducing infection and the spread of diseases. Medical professionals who work in medical settings frequently wash their hands to avoid spreading infective agents between patients or to themselves. Additionally, food preparation specialists must keep their clean hands so they do not put germs into the food they are making. Also, childcare workers must wash their hands often so they do not make children sick. It is also recognized that handling money, turning doorknobs, and coughing transfer bacteria and viruses to the skin, and especially the hands. These viruses are often spread to other people's hands, mouth, and eyes, which causes infection. Many of the diseases spread this way are more inconvenient than a simple cold, including the flu, hepatitis A, typhoid, and most recently—Coronavirus.

Generally, the conventional bar soap and liquid soap used for hand washing does not provide a visual indication as to whether the soap has been used for an appropriate amount of time for the process to be effective in removing germs. As a result, hands are often washed for too short an amount of time for the process to be effective in cleansing hands. Unfortunately, there is a tendency to hurry up the hand washing process and do a cursory job for only several seconds and this is believed inadequate. Failure to wash hands adequately may result in the spreading of colds, diarrhea, and other infectious diseases such as meningitis, flu and other viruses.

Further, water alone may rinse off dirt, but germs, such as viruses and bacteria, are so small they require chemical and mechanical intervention to get their sticky nanoparticles out of the crevices that make up our unique fingerprints. That's why thorough washing with soap is imperative to remove such germs. By washing the hands with soap for 20 seconds, at least, the nonpolar tail of soap penetrates the oily lipid membrane found in bacteria and viruses. This is similar to a nail popping a tire, where the water-repelling end of the soap molecule, a hydrophobic tail that can bond with oil and fats, stabs COVID-19 and leaves the virus a deflated and broken sack of RNA cells.

It is recognized that the capacity of soap to kill germs in such a manner is due to the dual nature of soap molecules. As the hydrophilic, or water-loving, heads reach out to bond with the water, the tails turn inwards to protect themselves from the water and by doing so, scoop up anything they catch in tiny soap bubble cages called micelles. Scrubbing all parts of the hands and wrists vigorously, with a sudsy lather, is key to locking the germs away—and washing them down the drain.

Other proposals have involved hand washing systems with visual indicators to help in determining if and when the germs are removed from the skin. The problem with these hand washing systems is that they do not provide a color indication of when 20 seconds has elapsed, so as to indicate that the germs are killed and dissolved. Also, the use of a moisturizer to hydrate the skin is not disclosed with the skin washing systems. Even though the above cited skin washing systems meet some of the needs of the market, a skin cleansing system and method for visual indication of an efficacious cleansing duration that provides a cleaning composition comprising soap and a colored moisturizer, which works to kill germs; and then visually indicate, through color, when a sufficient duration of approximately 20 seconds of cleansing has occurred, is still desired.

### SUMMARY

Illustrative embodiments of the disclosure are generally directed to a skin cleansing system and method for visual indication of an efficacious cleansing duration. The skin cleansing system and method provides a cleaning composition that works to kill germs; and then visually indicate, through color, when a sufficient cleaning duration of cleansing has occurred by simultaneously washing away a coloring agent in the moisturizer.

The cleaning composition comprises a liquid soap and a colored moisturizer that remain separated in a dispenser, but combine simultaneously when applied to the skin for washing with water. The soap requires a cleaning duration of about 20 seconds to kill the germs. The soap also requires the same cleaning duration of 20 seconds to dissolve the colored moisturizer. Thus, when the colored moisturizer washes away with water, this is an indication that the germs have also been dissolved and washed away. The dissolving action is a result of the nonpolar tail of the soap engaging and destroying, both the germs and the nonpolar oils in the colored moisturizer. The dispenser segregates the soap and the colored moisturizer until dispensing, so that the soap does not dissolve the nonpolar oils of the moisturizer prior to the cleaning process.

In some embodiments, the cleaning composition comprises a soap having a hydrophilic, or polar head; and an opposing hydrophobic, or nonpolar tail. The nonpolar tail is known in the art to bond with oils and fats, and when encountering a germ, such as a virus or bacteria, penetrate the virus or bacteria, so as to leave the virus or bacteria a deflated and broken sack of RNA cells. The polar, or hydrophilic head of the soap is known in the art to be soluble in water, and thereby help lift the germs away from the skin during washing. Furthermore, as the polar/hydrophilic heads reach out to bond with the water, the nonpolar/hydrophobic tails turn inwards to protect themselves from the water and by doing so, scoop up the germs, they catch in tiny soap bubble cages called micelles.

It is also known in the art that the polar head of the soap bonds to other polar molecules, such as nonpolar oils commonly found in moisturizers. Thus, the cleaning composition also includes a colored moisturizer. The colored moisturizer comprises at least a nonpolar oil and a coloring agent. The nonpolar oil of the colored moisturizer is susceptible to being dissolved and destroyed by the nonpolar tail of the soap—similar to the germs.

Furthermore, the soap and colored moisturizer are segregated in a dispenser until dispensing for cleaning the skin. This separation of components is necessary to prevent the soap from destroying the nonpolar oils of the colored moisturizer before the cleaning process begins. The soap and colored moisturizer can be combined through various manipulations and agitating motions of the dispenser. For example, the dispenser can have two separate chambers in fluid communication at a neck that extends to an outlet hole. The soap and colored moisturizer mix at the neck when the dispenser is rotated, and gravity carries the mixed combination onto the skin for cleaning. However, other mixing and dispensing means known in the art may also be used.

When the soap and the colored moisturizer are applied to the skin for cleaning, the nonpolar tail of the soap requires about 20 seconds to bond with, dissolve, and destroy the germs; and a similar 20 seconds to bond with, dissolve, and destroy the nonpolar oil of the colored moisturizer. When destroyed by the nonpolar tails of the soap, these two nonpolar components (germs and moisturizer) dissolve into the water that is being used to wash the skin. Consequently, as the colored moisturizer and the germs simultaneously dissolve into the water, the coloring agent in the colored moisturizer follows suit, and is no longer visible on the skin. In essence, 20 seconds after all germs have been dissolved, the color indication from the colored moisturizer is also dissolved.

In one embodiment, a skin cleansing system for visual indication of an efficacious cleansing duration, comprises a dispenser defined by a sidewall forming an interior cavity, an outlet port, and a divider traversing the interior cavity, whereby two independent compartments form in the interior cavity, the compartments being in fluid communication with the outlet port, the dispenser operable to combine the contents of each compartment at the outlet port during dispensing of the contents of each compartment.

In another embodiment, the skin cleansing system for visual indication of an efficacious cleansing duration, comprises a soap disposed in the first compartment of the dispenser, the soap being defined by a molecular structure comprising a polar head and a nonpolar tail.

In another embodiment, the skin cleansing system for visual indication of an efficacious cleansing duration, comprises a colored moisturizer disposed in the second compartment of the dispenser, the colored moisturizer comprising a nonpolar oil and a coloring agent; whereby, when the soap and the colored moisturizer are rubbed on the skin with water for a predetermined cleaning duration, the nonpolar tail of the soap engages and kills a germ, and the nonpolar tail of the soap engages and breaks up the nonpolar oil of the colored moisturizer, whereby the water dissolves and carries away the germ and the coloring agent of the colored moisturizer.

In another aspect, the predetermined cleaning duration is approximately 20 seconds.

In another aspect, the dispenser comprises a spin mechanism to mount to a surface.

In another aspect, the spin mechanism is a suction cup.

In another aspect, the dispenser is operable to spin 360°, in order to combine the soap and the colored moisturizer.

In another aspect, the dispenser comprises a pump mechanism in fluid communication with the outlet port.

In another aspect, the dispenser is defined by an hourglass shape.

In another aspect, the soap comprises a liquid soap.

In another aspect, the soap comprises a soap fragrance.

In another aspect, the coloring agent is integral with the nonpolar oil of the colored moisturizer.

In another aspect, the colored moisturizer comprises a skin hydrator and a moisturizer fragrance.

In another aspect, the nonpolar oil of the colored moisturizer includes at least one of the following: lanolin, mineral oil, petrolatum, castor oil, silicone oil, and cocoa butter.

In another aspect, the system is operable for washing the skin of the hands.

In another aspect, the when the soap and the colored moisturizer are rubbed on the skin with the water flowing from a faucet.

One objective of the present invention is to provide a visual indicator, during the washing of the hands, to help in determining if and when the germs are removed from the skin

Another objective is to segregate the soap and the colored moisturizer in the dispenser, until dispended for washing purposes.

An exemplary objective is to provide a dispenser that is mechanically simple, and maintains segregation between soap and moisturizer until dispensed.

Yet another objective is to provide a chemical-free cleaning composition.

An exemplary objective is to provide a colored moisturizer that inhibits dry skin.

Additional objectives are to provide an inexpensive to manufacture cleaning composition, and easy to use process for application thereof.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a Table listing the ingredients used for the cleaning composition for an exemplary skin cleansing system for visual indication of an efficacious cleansing duration, in accordance with an embodiment of the present invention;

FIG. 2 illustrates a diagram of an exemplary germ as a virus, showing the virus molecular makeup, in accordance with an embodiment of the present invention;

FIG. 3 illustrates a diagram of an exemplary soap molecule killing the virus, showing the virus molecular makeup, in accordance with an embodiment of the present invention;

FIG. 4 illustrates a diagram of exemplary soap micelles trapping virus proteins and water dissolving and carrying away the virus proteins, in accordance with an embodiment of the present invention;

FIG. 5 illustrates a front view of the dispenser, in accordance with an embodiment of the present invention;

FIG. 6 illustrates a sectioned view of the dispenser shown in FIG. 5 containing a liquid soap and a colored moisturizer in separate compartments, in accordance with an embodiment of the present invention;

FIG. 7 illustrates an elevated side view of the dispenser shown in FIG. 5, in accordance with an embodiment of the present invention; and

FIG. 8 illustrates a flowchart of an exemplary skin cleansing method for visual indication of an efficacious cleansing duration, in accordance with an embodiment of the present invention.

Like reference numerals refer to like parts throughout the various views of the drawings.

### DETAILED DESCRIPTION OF THE INVENTION

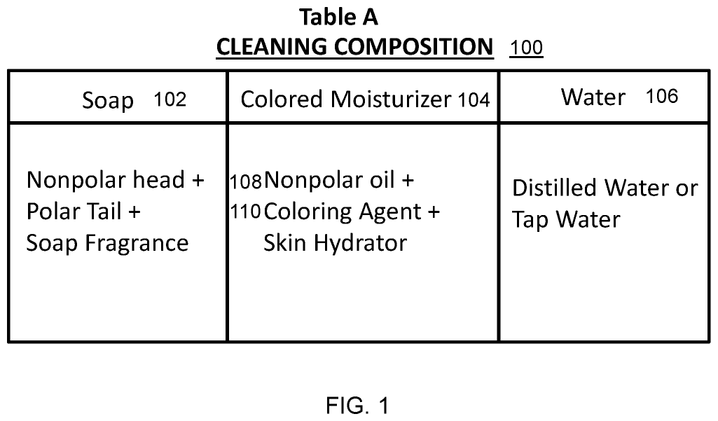

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

A skin cleansing system 600 and method 800 for visual indication of an efficacious cleansing duration is referenced in FIGS. 1-8. The skin cleansing system 600, hereafter “system 600”, leverages the molecular composition of a liquid soap 102 and a colored moisturizer 104 to produce a visual verification—color indicator—that a predetermined cleaning duration has elapsed during the cleansing of the skin. The elapsed cleaning duration is an axiomatic indication that germs 200 on the skin are killed and dissolved—that is in approximately 20 seconds. The color indication is the resultant of the colored moisturizer 104 also dissolving in the same 20 second elapsed duration as the germs 200 being killed. This phenomenon is the result of nonpolar tail of the soap 102 engaging and eliminating both the germs 200 and the nonpolar oils of the colored moisturizer 104 in approximately the same 20 second period. In essence, when the colored moisturizer 104 washes away with water 106, this is an indication that the germs 200 have also been dissolved and washed away. The dissolving action is a result of the nonpolar tail of the soap 102 engaging and destroying, both the germs 200 and the nonpolar oils in the colored moisturizer 104.

As Table A in FIG. 1 references, a cleaning composition 100 comprises a soap 102 and a colored moisturizer 104, which are combinable and used with water 106 for cleaning the skin. For purposes of the present invention, the skin can include human or animal skin; and further comprises any part of the body. Though in one possible embodiment, the cleansing of the hands is referenced. Thus, soap 102, colored moisturizer 104, and water 106, when combined in a specific sequence and rubbed onto the skin for a specific cleaning duration, provide visual indication that the germ 200 has been removed from the skin.

In one non-limiting embodiment, the soap 102 comprises a liquid soap that easily flows from a dispenser 500. The soap 102 may also consist of a soap fragrance, a soap coloring agent, and various vitamins and hydrators known in the art of soaps. It is axiomatic that the soap 102 is defined by a molecular structure, i.e., soap molecule 300 having a hydrophilic, or polar head 302; and an opposing hydrophobic, or nonpolar tail 304.

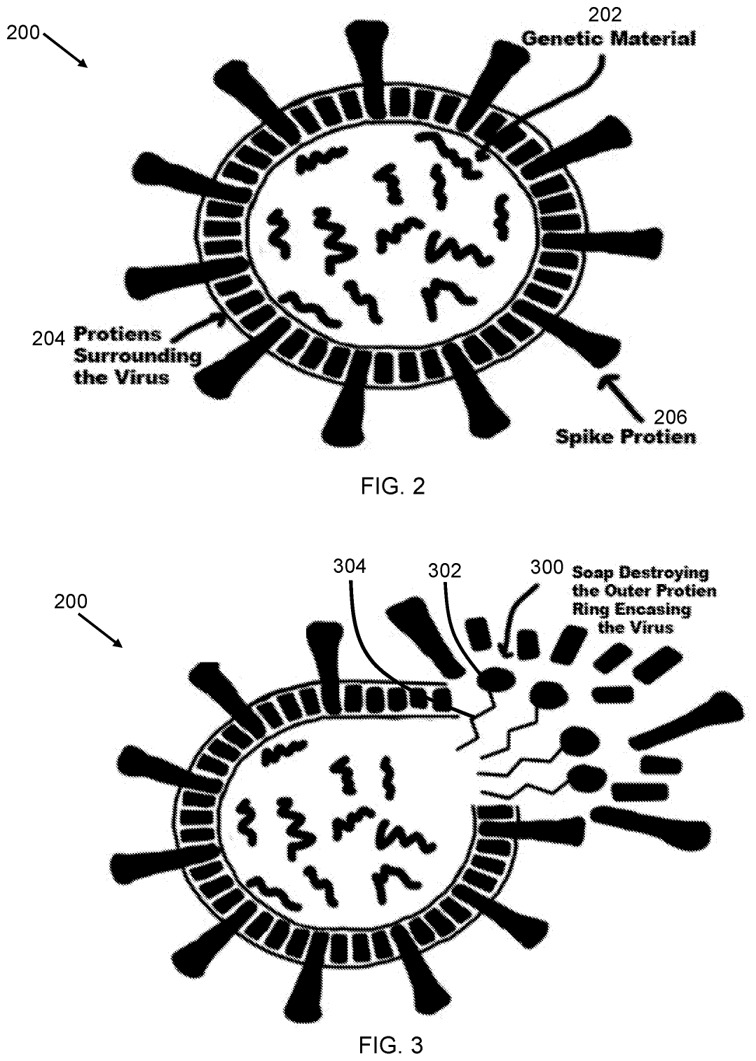

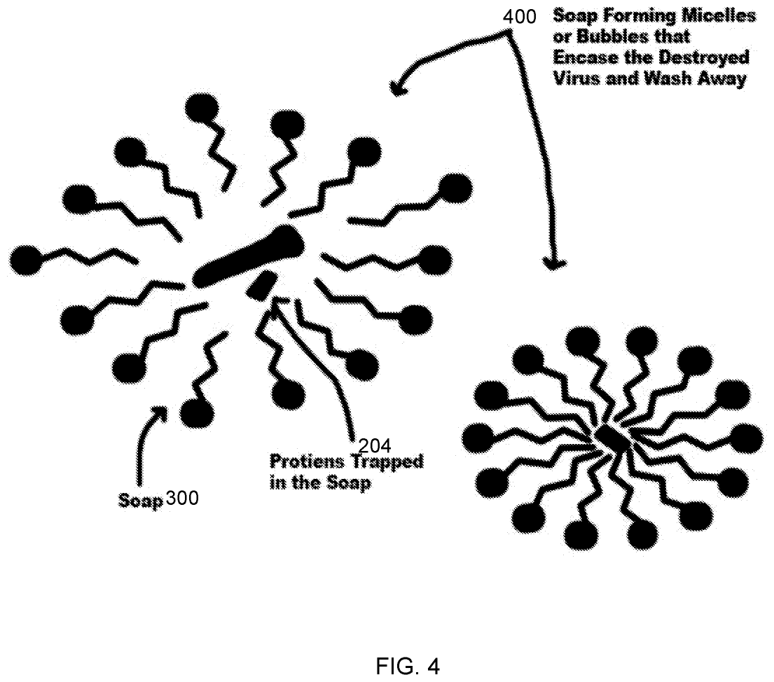

The nonpolar tail 304 of the soap 102 is known in the art to bond with oils and fats, and when encountering a germ, such as a virus or bacteria, penetrate the virus or bacteria. This results in the virus or bacteria being deflated and broken sack of RNA cells (See FIGS. 2 and 3). The nonpolar tail 304 of the soap 102 also serves to surround, and subsequently dissolve, nonpolar oils 108 commonly found in moisturizers.

Furthermore, the polar head 302 of the soap 102 is known in the art to be soluble in water 106, and thereby help lift the germs 200 away from the skin during washing. For example, FIG. 4 shows soap 102 micelles surrounding and washing away (dissolving) germ. Furthermore, as the polar/hydrophilic heads reach out to bond with the water 106, the nonpolar tails 304 turn inwards to protect themselves from the water 106 and by doing so, scoop up the germs, they catch in tiny soap 102 bubble cages, or soap micelles 400. It is the soap micelles 400 that carry away the broken proteins 204, 206 of the germ 200, and the nonpolar oils 108 and coloring agents 110 of the colored moisturizer 104.

It is also known in the art that the polar head 304 of the soap 102 bonds to other polar molecules, such as nonpolar oils commonly found in moisturizers. Thus, the cleaning composition 100 also includes a colored moisturizer 104. The colored moisturizer 104 serves the dual purpose of moisturizing the skin, and combining with the soap 102 to create visual, color indication that a cleaning duration of approximately 20 seconds has elapsed. In some embodiments, the colored moisturizer 104 consists of a skin hydrator and a moisturizer fragrance.

The colored moisturizer 104 comprises at least a nonpolar oil 108 and a coloring agent 110. The nonpolar oil 108 of the colored moisturizer 104 may include, without limitation, lanolin, mineral oil, petrolatum, castor oil, silicone oil, and cocoa butter. It is axiomatic that the nonpolar oil 108 of the colored moisturizer 104 is susceptible to being dissolved and destroyed by the nonpolar tail 304 of the soap 102—similar to the germs. The coloring agent 110 is integral with the nonpolar oil 108 of the colored moisturizer 104. In this manner, as the nonpolar oil 108 dissolves, the coloring agent 110 follows suit, and is washed away by the water 106.

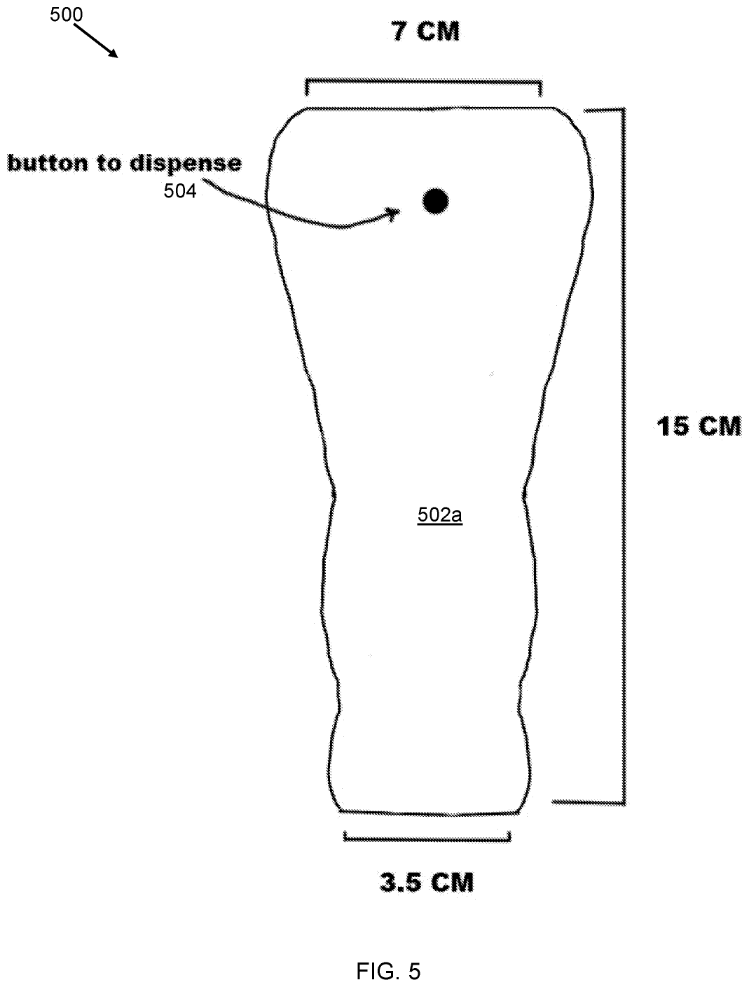

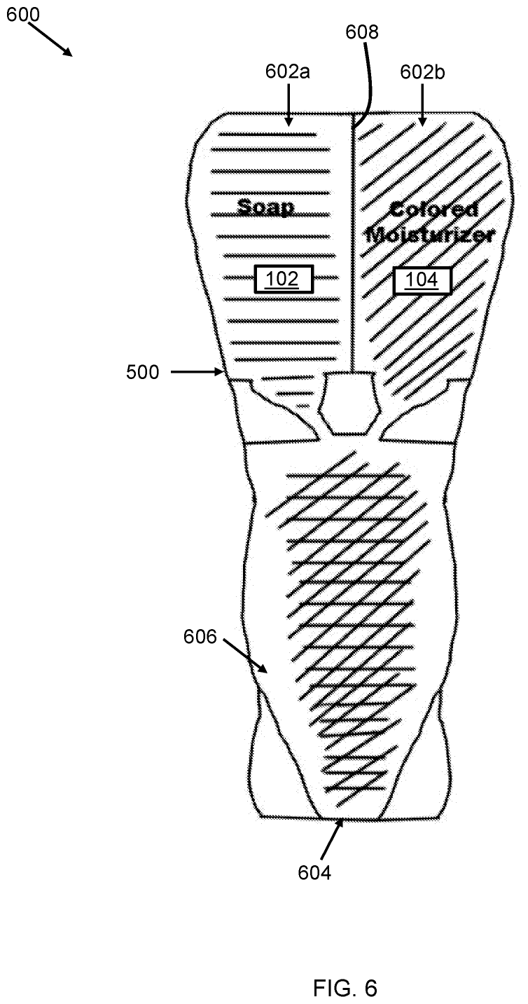

Looking now at dispenser 500 illustration in FIG. 5, the soap 102 and colored moisturizer 104 are segregated in a dispenser 500 until dispensing for cleaning the skin. This separation of the soap 102 and colored moisturizer 104 in separate compartments 502a-n is necessary to prevent the soap 102 from destroying the nonpolar oil 108 of the colored moisturizer 104 before the cleaning process begins. For example, FIG. 6 illustrates a sectioned view of the dispenser 500 containing a liquid soap and a colored moisturizer 104 in separate compartments. From the dispenser 500, the soap 102 and colored moisturizer 104 can be combined through various manipulations and agitating motions.

In some embodiments, the dispenser 500 is defined by multiple sidewalls 502a, 502n forming an interior cavity 606, an outlet port 604, and a divider 608 traversing the interior cavity 606. The divider 608 creates a structural configuration in which at least two independent compartments 602, 602b form in the interior cavity 606. The compartments 602a-b are in fluid communication with the outlet port 604. In one non-limiting embodiment, the compartments 602a-b are equal in volume.

The dispenser 500 is configured to combine the contents of each compartment 602a-b at the outlet port 604 during dispensing of the contents of each compartment. For example, the dispenser 500 can have two separate chambers in fluid communication at a neck that extends to an outlet hole. The soap 102 and colored moisturizer 104 are dispensed, so as to mix at the neck when the dispenser 500 is rotated, and gravity carries the mixed combination onto the skin for cleaning. However, other mixing and dispensing means known in the art may also be used.

It is significant to note that the soap 102 and colored moisturizer 104 cannot be allowed to mix before the washing process, or dispensing. This is because the nonpolar tail 304 of the soap 102 engages and surrounds the nonpolar oil 108 of the colored moisturizer 104, which would destroy the molecular composition of the colored moisturizer 104 prior to being dispensed on the hands. Thus, the segregation of the soap 102 and the colored moisturizer 104 is necessary in separate compartments 602a, 602b of the dispenser 500.

When the soap 102 and the colored moisturizer 104 are combined during dispensing; and rubbed on the skin with water 106 for a predetermined cleaning duration: the nonpolar tail 304 of the soap 102 engages and kills a germ 200, and the nonpolar tail 304 of the soap 102 engages and breaks up the nonpolar oil 108 of the colored moisturizer 104. It is axiomatic that the nonpolar tail 304 of the soap 102 requires about 20 seconds to bond with, dissolve, and destroy the germs 200; and a similar 20 seconds to bond with, dissolve, and destroy the nonpolar oil 108 of the colored moisturizer 104. Thus, when the germs 200 are killed and washed away, the colored moisturizer 104, including the coloring agent 110, is also dissolved and washed away by the water 106.

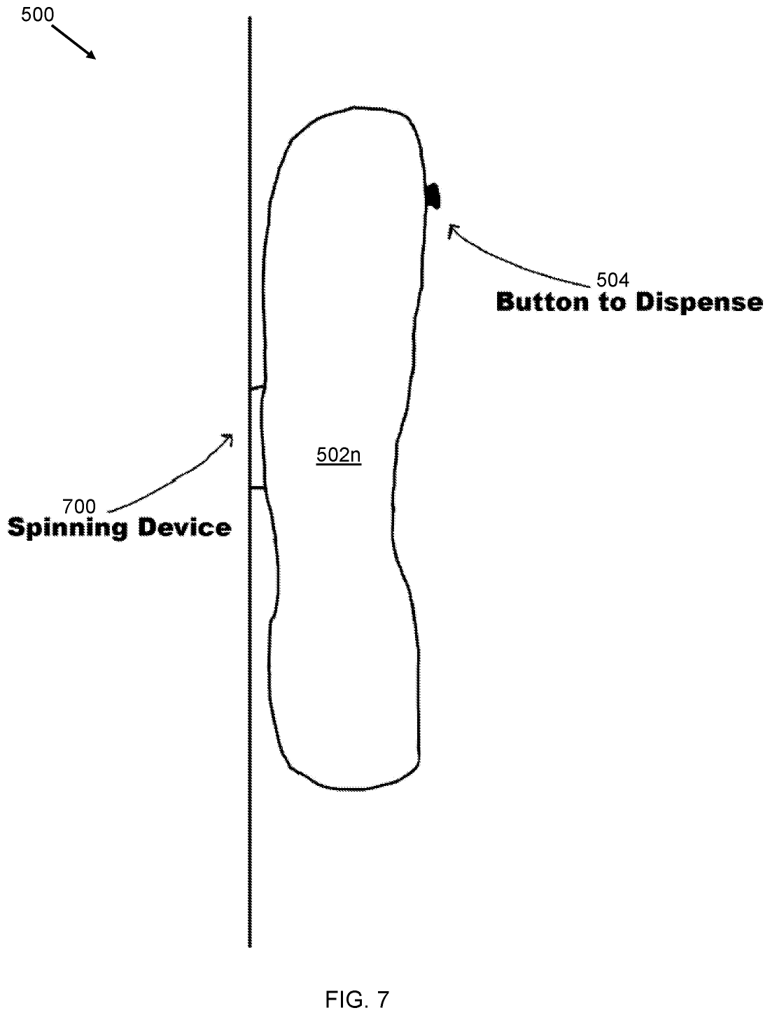

In one non-limiting embodiment shown in FIG. 7, the dispenser 500 comprises a spin mechanism that enables mounting of the dispenser 500 to a mounting surface. This can include a suction cup with a rotatable base. The spin mechanism 700, may also be a rotatable mount that enables the dispenser 500 to spin; and thereby combine and dispense the soap 102 and colored moisturizer 104. In some embodiments, the spin mechanism 700 enables the dispenser 500 to spin 360°, in order to combine the soap 102 and the colored moisturizer 104.

In yet other embodiments, the dispenser 500 further comprises a pump mechanism 504 that is in fluid communication with the outlet port 604. The pump mechanism can include a dispensing button that creates a suction effect to dispense the combined soap 102 and colored moisturizer 104 (See FIG. 5). In one non-limiting embodiment, the dispenser 500 is defined by an hourglass shape. However, other designs, shapes, and material compositions may be used for the dispenser 500. Thus, the dispenser 500 achieves the objective of segregating the soap 102 and the colored moisturizer 104 in the dispenser 500, until combined and dispended for cleaning the skin with water 106.

FIG. 2 illustrates a diagram of an exemplary germ 200, such as a virus, showing the virus molecular makeup. The center of the virus has the generic makeup 202, i.e., DNA, RNA. The outer portion 204 of the virus surrounds the genetic makeup with a protein, which is suspectable to penetration by the nonpolar tail 304 of the soap 102. Further, the proteins can include spike proteins 206 that protrude from the outer surface of the virus. However, it is significant to note that the system 600 is efficacious in destroying germs 200 other than viruses. Any germ having a protein susceptible to attack by the nonpolar tail 304 of the soap 102 is a target of the nonpolar tail 304 of the soap 102; and consequently, susceptible to being washed away with the moisturizer 104.

FIG. 3 illustrates a diagram of an exemplary soap 102 molecule 300 killing the virus, showing the virus molecular makeup. The nonpolar tail 304 of the soap 102 destroys the outer surface proteins through a vicious penetrating attack. Continuing, FIG. 4 illustrates a diagram of exemplary soap micelles 400 trapping the virus proteins, and water 106 dissolving and carrying away the virus proteins 204, 206.

Simultaneously, the soap micelles 400 and water 106 wash away the nonpolar oil 108 of the colored moisturizer 104, and consequently the coloring agent 110 of the colored moisturizer 104. The process takes about 20 seconds for the soap 102 molecules to kill and wash away the virus and the colored moisturizer 104. Thus, as the coloring agent 110 is washed away, this is indicative that the germ has also been killed and washed away.

When destroyed by the nonpolar tail 304 of the soap 102, these two nonpolar components (germs and moisturizer) dissolve into the water 106 that is being used to wash the skin. In this manner, the water 106 dissolves and carries away the germ and the coloring agent 110 of the colored moisturizer 104. Consequently, as the colored moisturizer 104 and the germs simultaneously dissolve into the water 106, the coloring agent 110 in the colored moisturizer 104 follows suit, and is no longer visible on the skin.

In essence, 20 seconds after all germs 200 have been dissolved, the color indication from the colored moisturizer 104 is also dissolved. As the nonpolar oil 108 dissolves, the coloring agent 110 follows suit, and is washed away by the water 106. This achieves the primary objective of providing a visual indicator, during the washing of the hands, to help in determining if and when the germs 200 are removed from the skin.

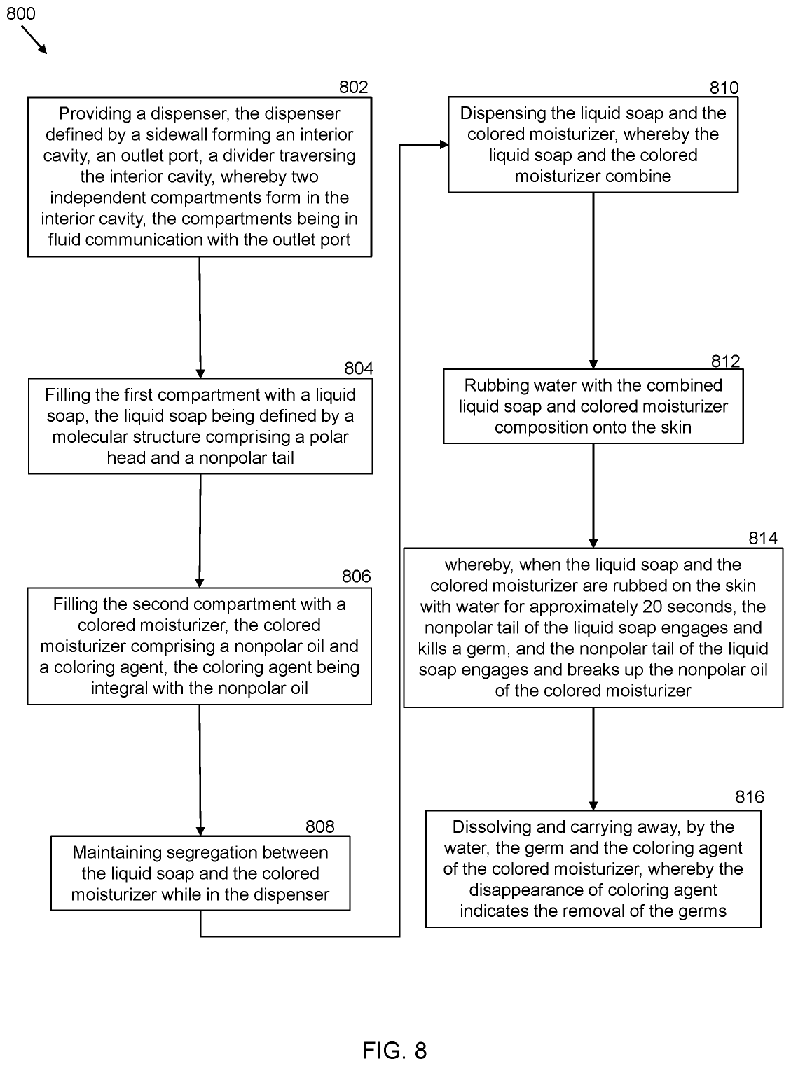

FIG. 8 references a flowchart for an exemplary skin cleansing method 800 for visual indication of an efficacious cleansing duration. The method 800 may include an initial Step 802 of providing a dispenser, the dispenser 500 defined by multiple sidewalls forming an interior cavity, an outlet port, a divider traversing the interior cavity, whereby two independent compartments form in the interior cavity, the compartments being in fluid communication with the outlet port. The compartments 602a-b are necessary to segregate the soap 102 and colored moisturizer 104 until dispensing on the skin with water 106.

The method 800 may further comprise a Step 804 of filling the first compartment with a liquid soap, the liquid soap being defined by a molecular structure comprising a polar head and a nonpolar tail. A Step 806 includes filling the second compartment with a colored moisturizer, the colored moisturizer comprising a nonpolar oil and a coloring agent, the coloring agent being integral with the nonpolar oil. In some embodiments, a Step 808 comprises maintaining segregation between the liquid soap and the colored moisturizer while in the dispenser. A Step 810 includes dispensing the liquid soap and the colored moisturizer, whereby the liquid soap and the colored moisturizer combine.

In some embodiments, a Step 812 may include rubbing water with the combined liquid soap and colored moisturizer composition onto the skin. A Step 814 comprises whereby, when the liquid soap and the colored moisturizer are rubbed on the skin with water for approximately 20 seconds, the nonpolar tail of the liquid soap engages and kills a germ, and the nonpolar tail of the liquid soap engages and breaks up the nonpolar oil of the colored moisturizer. A final Step 816 includes dissolving and carrying away, by the water, the germ and the coloring agent of the colored moisturizer, whereby the disappearance of coloring agent indicates the removal of the germs.

Although the process-flow diagrams show a specific order of executing the process steps, the order of executing the steps may be changed relative to the order shown in certain embodiments. Also, two or more blocks shown in succession may be executed concurrently or with partial concurrence in some embodiments. Certain steps may also be omitted from the process-flow diagrams for the sake of brevity. In some embodiments, some or all the process steps shown in the process-flow diagrams can be combined into a single process.

These and other advantages of the invention will be further understood and appreciated by those skilled in the art by reference to the following written specification, claims and appended drawings.

In conclusion, a skin cleansing system 600 and method 800 for visual indication of an efficacious cleansing duration provides a cleaning composition 100 that works to kill germs; and then visually indicate, through color, when a sufficient cleaning duration, of approximately 20 seconds, of cleansing has occurred. The cleaning composition 100 comprises a liquid soap 102 and a colored moisturizer 104 that remain separated in a dispenser, but combine simultaneously when applied to the skin for washing with water 106. The soap 102 requires a cleaning duration of about 20 seconds to kill the germs. The soap also requires the same cleaning duration of 20 seconds to dissolve the nonpolar oils in the colored moisturizer. Thus, when the colored moisturizer 104 washes away, this is an indication that the germs have also washed away. The dissolving action is a result of the nonpolar tail 304 of the soap 102 engaging and destroying, both the germs 200 and the nonpolar oils 108 in the colored moisturizer 104.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A skin cleansing system for visual indication of an efficacious cleansing duration, the system comprising:
a dispenser defined by multiple sidewalls forming an interior cavity, the dispenser further being defined an outlet port, and a divider traversing the interior cavity,
whereby two independent compartments form in the interior cavity, the compartments being in fluid communication with the outlet port, the dispenser operable to combine the contents of each compartment at the outlet port during dispensing of the contents of each compartment;
a soap disposed in the first compartment of the dispenser, the soap being defined by a molecular structure comprising a polar head and a nonpolar tail; and
a colored moisturizer disposed in the second compartment of the dispenser, the colored moisturizer comprising a nonpolar oil and a coloring agent,
the colored moisturizer and the soap combining at the outlet port when dispensed from the dispenser,
whereby, when the soap and the colored moisturizer are rubbed on the skin with water for a predetermined cleaning duration, the nonpolar tail of the soap engages and kills a germ, and the nonpolar tail of the soap engages and breaks up the nonpolar oil of the colored moisturizer,
whereby the water dissolves and carries away the germ and the coloring agent of the colored moisturizer.

2. The system of claim 1, wherein the predetermined cleaning duration is approximately 20 seconds.

3. The system of claim 1, wherein the dispenser comprises a spin mechanism to mount the dispenser to a surface and rotate the dispenser.

4. The system of claim 3, wherein the spin mechanism comprises a suction cup.

5. The system of claim 1, wherein the dispenser is operable to spin 360 degrees, in order to combine the soap and the colored moisturizer.

6. The system of claim 1, wherein the dispenser comprises a pump mechanism in fluid communication with the outlet port.

7. The system of claim 1, wherein the dispenser is defined by an hourglass shape.

8. The system of claim 1, wherein the soap comprises a liquid soap.

9. The system of claim 1, wherein the soap comprises a soap fragrance.

10. The system of claim 1, wherein the coloring agent is integral with the nonpolar oil of the colored moisturizer.

11. The system of claim 1, wherein the colored moisturizer comprises a skin hydrator and a moisturizer fragrance.

12. The system of claim 1, wherein the nonpolar oil of the colored moisturizer includes at least one of the following: lanolin, mineral oil, petrolatum, castor oil, silicone oil, and cocoa butter.

13. The system of claim 1, wherein the system is operable for washing the skin of the hands.

14. The system of claim 1, wherein the germ includes at least one of the following: a bacterium, a virus, a fungus, and a protozoon.

15. A skin cleansing system for visual indication of an efficacious cleansing duration, the system comprising:
a dispenser defined by multiple sidewalls forming an interior cavity, the dispenser further being defined by an outlet port, a divider traversing the interior cavity, and a pump mechanism in fluid communication with the outlet port,
whereby two independent compartments form in the interior cavity, the compartments being in fluid communication with the outlet port, the dispenser operable to combine the contents of each compartment at the outlet port during dispensing of the contents of each compartment;
a liquid soap disposed in the first compartment of the dispenser, the liquid soap being defined by a molecular structure comprising a polar head and a nonpolar tail; and
a colored moisturizer disposed in the second compartment of the dispenser, the colored moisturizer comprising a nonpolar oil and a coloring agent, the coloring agent being integral with the nonpolar oil,
the colored moisturizer and the liquid soap combining at the outlet port when dispensed from the dispenser,
whereby, when the liquid soap and the colored moisturizer are rubbed on the skin with water for approximately 20 seconds, the nonpolar tail of the liquid soap engages and kills a germ, and the nonpolar tail of the liquid soap engages and breaks up the nonpolar oil of the colored moisturizer,
whereby the water dissolves and carries away the germ and the coloring agent of the colored moisturizer.

16. The system of claim 15, wherein the dispenser comprises a spin mechanism to mount the dispenser to a surface and rotate the dispenser.

17. The system of claim 15, wherein the dispenser is operable to spin 360 degrees, in order to combine the soap and the colored moisturizer.

18. The system of claim 15, wherein the colored moisturizer comprises a skin hydrator and a moisturizer fragrance.

19. The system of claim 15, wherein the nonpolar oil of the colored moisturizer includes at least one of the following: lanolin, mineral oil, petrolatum, castor oil, silicone oil, and cocoa butter.

20. A skin cleansing method for visual indication of an efficacious cleansing duration, the method comprising:
providing a dispenser, the dispenser defined by a sidewall forming an interior cavity, an outlet port, a divider traversing the interior cavity, whereby two independent compartments form in the interior cavity, the compartments being in fluid communication with the outlet port;
filling the first compartment with a liquid soap, the liquid soap being defined by a molecular structure comprising a polar head and a nonpolar tail; and
filling the second compartment with a colored moisturizer, the colored moisturizer comprising a nonpolar oil and a coloring agent, the coloring agent being integral with the nonpolar oil;
maintaining segregation between the liquid soap and the colored moisturizer while in the dispenser;
dispensing the liquid soap and the colored moisturizer, whereby the liquid soap and the colored moisturizer combine;
rubbing water with the combined liquid soap and colored moisturizer composition onto the skin,
whereby, when the liquid soap and the colored moisturizer are rubbed on the skin with water for approximately 20 seconds, the nonpolar tail of the liquid soap engages and kills a germ, and the nonpolar tail of the liquid soap engages and breaks up the nonpolar oil of the colored moisturizer; and
dissolving and carrying away, by the water, the germ and the coloring agent of the colored moisturizer, whereby the disappearance of coloring agent indicates the removal of the germs.
